(19) United States
(12) Patent Application Publication
(10) Pub. No.: US 2025/0127515 A1
(43) Pub. Date: Apr. 24, 2025

(54) MULTI-LUMEN CATHETER WITH OCCLUSION ELEMENT

(71) Applicant: ABIOMED, Inc., Danvers, MA (US)

(72) Inventor: Anthony Wong, Danvers, MA (US)

(73) Assignee: ABIOMED, Inc., Danvers, MA (US)

(21) Appl. No.: 18/922,641

(22) Filed: Oct. 22, 2024

Related U.S. Application Data
(60) Provisional application No. 63/545,303, filed on Oct. 23, 2023.

(51) Int. Cl. A61B 17/12; A61B 17/00; A61B 90/00

(52) U.S. Cl. CPC A61B 17/12036; A61B 17/00234; A61B 17/12109; A61B 17/12136; A61B 90/06; A61B 2017/00022; A61B 2017/00305; A61B 2017/00557; A61B 2017/00561; A61B 2017/00862; A61B 2090/064

(57) ABSTRACT

A catheter may be provided. The catheter may include an expandable occlusion element coupled to an external surface of a tubular body at a proximal end of the expandable occlusion element and a distal end of the expandable occlusion element. An inner surface of the expandable occlusion element may define a volume of space between the inner surface of the expandable occlusion element and an external surface of the tubular body. The catheter may include a plurality of lumens. A first lumen may be configured to allow a first fluid to be introduced and/or removed from the volume of space defined by the inner surface of the expandable occlusion element. A second lumen may be configured to receive a pressure sensor or define a fluid-filled pressure transducer. A third lumen may be configured to allow a second fluid to be removed from the volume of space.

### CROSS-REFERENCE TO RELATED APPLICATIONS

The present application claims priority to U.S. Provisional Application No. 63/545,303, filed on Oct. 23, 2023, which is incorporated by reference herein by its entirety.

### TECHNICAL FIELD

The present disclosure is drawn to multi-lumen catheters, and specifically, to multi-lumen catheters used on devices for cardiovascular treatments with occlusion elements.

### BACKGROUND

If the pumping function of a patient's heart is insufficient despite other medical treatments, the circulatory system can be assisted in various ways. In some cases, a flow-restricting device (which may include, e.g., an inflatable balloon) may be installed into the patient's superior vena cava (SVC) to assist in regulating venous blood return to the heart. By adjusting the flow-restrictive device, the amount of blood flowing back to the heart can be controlled.

### BRIEF SUMMARY

In various aspects, a catheter may be provided. The catheter may include an expandable occlusion element (which may be, e.g., a balloon). The expandable occlusion element may be coupled to an external surface of a tubular body. In particular, a proximal end of the expandable occlusion element and a distal end of the expandable occlusion element may be coupled to the tubular body. An inner surface of the expandable occlusion element may define a volume of space between the inner surface of the expandable occlusion element and an external surface of the tubular body.

The catheter may include a plurality of lumens. Each of the plurality of lumens may extend from a proximal end of the tubular body to an external surface of the tubular body at one or more defined exit points between the proximal end of the expandable occlusion element and the distal end of the expandable occlusion element. The plurality of lumens may have a first lumen. The first lumen may be configured to allow a fluid (which may be a liquid, such as saline fluid) to be introduced and/or removed from the volume of space defined by the inner surface of the expandable occlusion element. The plurality of lumens may include a second lumen. A second lumen may be configured to receive a pressure sensor or define a fluid-filled pressure transducer. The plurality of lumens may include a third lumen. The third lumen may be configured to allow air to be removed from the volume of space.

The first lumen may be operably coupled to a plurality of defined fluid exit points. Each of the defined fluid exit points may be disposed an axial distance from an adjacent defined fluid exit point. The plurality of defined fluid exit points may be three exit points. All defined fluid exit points may be circumferentially aligned. At least one defined fluid exit point may be rotated circumferentially, relative to an adjacent defined fluid exit point by an angle A1. In some embodiments, A1 may be greater than 0° and less than or equal to 45°.

In some embodiments, a defined exit point of the second lumen may be centered axially within the volume of space defined by the inner surface of the expandable occlusion element. The defined exit point of the second lumen may be rotated circumferentially, relative to any defined fluid exit point(s) of the first lumen, by an angle A2. In some embodiments, A2 may be greater than or equal to 75°. In some embodiments, A2 may be less than or equal to 105°.

In some embodiments, a defined exit point of the third lumen may be disposed axially between the defined exit point of the second lumen and the proximal end of the expandable occlusion element. The defined exit point of the third lumen may be rotated circumferentially, relative to any defined fluid exit points of the first lumen, by an angle A3. In some embodiments, A3 may be greater than or equal to 75°. In some embodiments, A3 may be greater than or equal to 150° and less than or equal to 180°.

The expandable occlusion element may include a conical distal end. The expandable occlusion element may include a conical proximal end. The expandable occlusion element may include a cylindrical intermediate portion between the distal end and the proximal end.

In some embodiments, the expandable occlusion element may define an angle A4 between the external surface of the tubular body and the conical proximal end when the expandable occlusion element is in its expanded configuration. In some embodiments, A4 may be greater than or equal to 30° and less than or equal to 60°.

In some embodiments, the expandable occlusion element may define an angle A5 between the external surface of the tubular body and the conical distal end when the expandable occlusion element is in its expanded configuration. In some embodiments, A5 may be greater than or equal to 30° and less than or equal to 60°. In some embodiments, A4 may equal A5. In some embodiments, A4 may be different from A5.

In some embodiments, an axial length of conical proximal end may be longer than an axial length of the cylindrical intermediate portion. An axial length of conical proximal end may be no longer than (e.g., short than or equal in length to) an axial length of the cylindrical intermediate portion.

In some embodiments, the second fluid may be a gas. In some embodiments, the second fluid may be air.

In various aspect a system may be provided. The system may include a catheter as disclosed herein. The system may include a controller operably coupled to the catheter. The system may include a vacuum pump operably coupled to third lumen. The system may include a sensor. The sensor may be operably coupled to the controller. The sensor may be configured to determine if fluid has passed from the expandable occlusion element through the third lumen. The sensor may be configured to determine how much fluid has passed from the expandable occlusion element through the third lumen.

In various aspects, a method for expanding an expandable occlusion element may be provided. The method may include providing a first fluid to the expandable occlusion element using one or more inlets and drawing a second fluid from the expandable occlusion element using an outlet. The method may further include measuring a pressure within the expandable occlusion element while providing the first fluid and determining if the first fluid has exited the outlet while providing the first fluid. The method may further include stopping withdraw of the second fluid after determining the first fluid has exited the outlet. The method may further include stopping fluid provision after determining the pressure has reached a predetermined threshold.

In certain embodiments, the method may further include determining how much first fluid has exited the outlet. In some embodiments, withdrawing the second fluid is stopped after a predetermined amount of first fluid has exited the outlet. In some embodiments, withdrawing the second fluid may be stopped, following a predetermined delay, after determining the first fluid has exited the outlet. In some embodiments, the first fluid may be a saline fluid. In some embodiments, the method may further include providing a contrast agent.

In some embodiments, the method may further include introducing the contrast agent to the first fluid. In some embodiments, drawing the second fluid may include withdrawing the second fluid from the expandable occlusion element intermittently. In some embodiments, drawing the second fluid includes withdrawing the second fluid from the expandable occlusion element continuously. In some embodiments, drawing the second fluid begins a predetermined period of time after the first fluid starts being provided to the expandable occlusion element.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1A is an illustration showing a device with a plurality of expandable occlusion elements within a patient.

FIG. 1B is a cross-sectional view of a catheter of the device in FIG. 1.

FIG. 2A is a side view of an embodiment of a catheter showing exit points for a first lumen.

FIG. 2B is a top-down view of a catheter with various exit points.

FIG. 3 is a side-view of exit points for a second lumen, relative to some first and third lumen.

FIG. 4 is a side-view of exit points for a third lumen, relative to some first and second lumen.

FIG. 5 is a perspective view of a catheter and a first occlusive element.

FIG. 6 is an illustration of a system.

FIG. 7 is a flowchart of a method.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

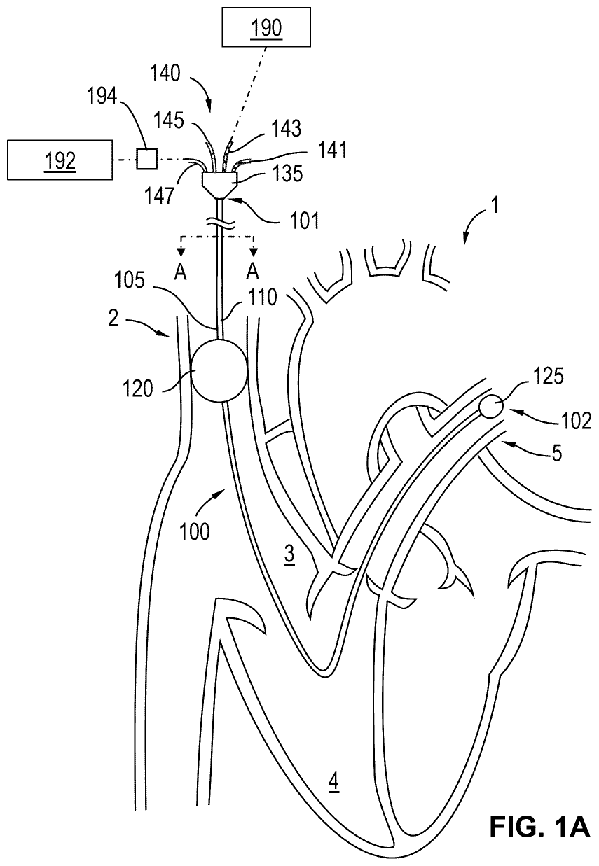

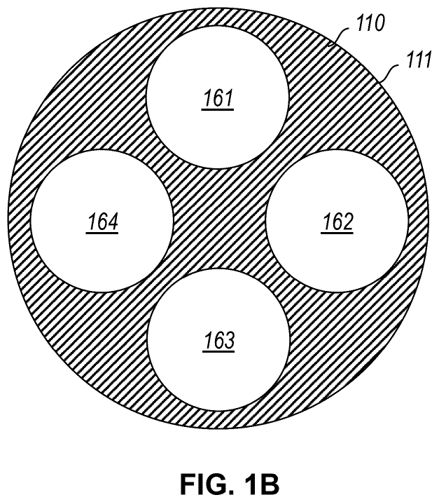

As seen in FIG. 1A, in some cardiovascular treatments, a flow-restrictive device 100 may be deployed at least partially within a patient. Here, the device is shown as being inserted into a patient's heart 1, extending through the superior vena cava 2, right atrium 3, right ventricle 4, and into the pulmonary artery 5. The device is shown as including a catheter 105. The catheter may have a tubular body 110 with a first end 101 and a second end 102. The device may be one or more occlusive elements (which may be, e.g., balloons or other similar known occlusion elements). The device is shown as having two expandable occlusion elements coupled to the catheter-a first expandable occlusion element 120 (shown here deployed in the superior vena cava, at an intermediate point on the catheter between a first end 101 and second end 102 of the catheter), and a second expandable occlusion element 125 (which here is deployed in the pulmonary artery, at a distal end of the catheter). As will be appreciated, the first expandable occlusion element 120 may be located anywhere inside of the patient. There may be a portion 135 of the catheter that extends outside the patient's body. As shown, to control the device, there may need to be a number of connections 140 to the catheter. Here, we see four connections, which may include, e.g., a guidewire 141, tubes 143 for connecting a fluid source 190 to the expandable occlusion element(s) (preferably one lumen per occlusion element), wires or tubes 145 for connecting to sensors in or on the device, and a tube 147 for withdrawing fluid (e.g., a liquid or gas) from one or both of the expandable occlusion elements (which may be operably coupled to a vacuum pump 192).

The expandable occlusion element may be coupled to an external surface of a tubular body. In some embodiments, a proximal end of the expandable occlusion element and a distal end of the expandable occlusion element may be coupled to the tubular body. An inner surface of the expandable occlusion element may define a volume of space between the inner surface of the expandable occlusion element and an external surface of the tubular body.

Controlling the expansion and contraction of occlusion elements on a flow-restrictive device can be challenging.

Referring to FIG. 1B, a cross-section of the catheter based on cutting plane A in FIG. 1 can be seen. As seen, multiple lumens may be present. A first lumen 161 may be configured to allow a fluid (which may be a liquid, such a saline fluid) to be introduced and/or removed from the volume of space defined by the inner surface of the expandable occlusion element. A second lumen 162 may be configured to receive a pressure sensor or define a fluid-filled pressure transducer. A third lumen 163 may be configured to allow air to be removed from the volume of space. A fourth lumen 164 may be configured to receive a guidewire. One or more additional lumen may be utilized. The lumen may be of any suitable cross-sectional geometry. For example, the lumens may have circular cross-sectional geometry (as shown in FIG. 1B).

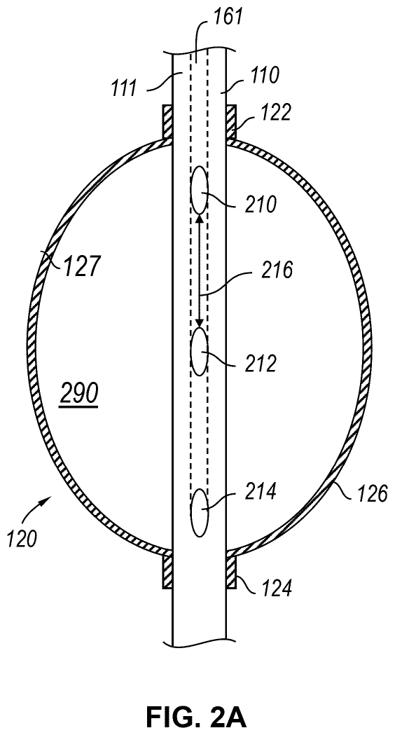

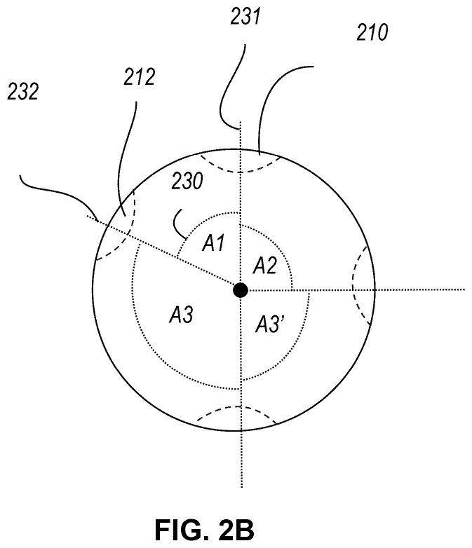

Referring to FIG. 2A, the occlusive element may be seen. A proximal portion 122 and a distal portion 124 may be coupled to an external surface 111 of the tubular body. An intermediate portion 126 may be disposed between the proximal and distal portions. An inner surface 127 of the occlusive element may form a volume of space 290 between the inner surface 127 and the external surface 111 of the tubular body 110. This volume of space will expand and contract to occlude (or not occlude) a blood vessel as needed.

The first lumen 161 may be operably coupled to a plurality of defined fluid exit points 210, 212, 214, each defined fluid exit point being disposed at an external surface 111 of the catheter 105 an axial distance 216 from an adjacent defined fluid exit point. As shown, the plurality of defined fluid exit points may be three exit points. As shown, all defined fluid exit points may be circumferentially aligned. Referring to FIG. 2B, at least one defined fluid exit point may be rotated circumferentially, relative to an adjacent defined fluid exit point by an angle 230 A1. The angle may be formed, when viewing down the central axis of the tubular member, between a centerline 231 through a first defined fluid exit point (here, defined fluid exit point 210) and a centerline 232 of a second defined fluid exit point (here, defined exit point 212). In some embodiments, A1 may be greater than 0°. In some embodiments, A1 may be less than or equal to 45°. In some embodiments, A1 may be less than or equal to 40°. In some embodiments, A1 may be less than or equal to 35°. In some embodiments, A1 may be less than or equal to 30°. In some embodiments, A1 may be less than or equal to 25°. In some embodiments, A1 may be less than or equal to 20°. In some embodiments, A1 may be less than or equal to 15°. In some embodiments, A1 may be less than or equal to 10°. In some embodiments, A1 may be less than or equal to 5°. One or more of the fluid exit points may be rotated circumferentially.

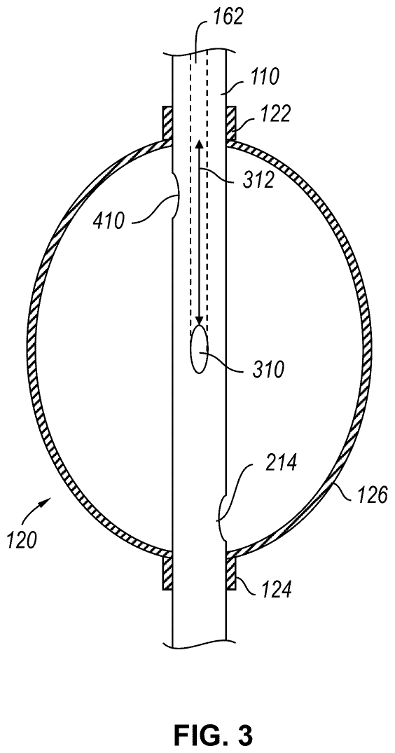

Referring to FIG. 3, a defined exit point 310 of the second lumen 162 may be disposed a distance 312 from the proximal end of the occlusive element. In some embodiments, the defined exit point 310 may be centered axially within the volume of space 290 defined by the inner surface of the expandable occlusion element. In some embodiments, the distance 312 may be less than or equal to 50% of the total axial length of the intermediate portion. In some embodiments, the distance may more than or equal to 50% of the total axial length of the intermediate portion.

Similar to what is shown in FIG. 2B, the defined exit point of the second lumen may be rotated circumferentially, relative to any defined fluid exit point(s) of the first lumen, by an angle A2. In some embodiments A2 equals 90°. In some embodiments, A2 may be greater than or equal to 75°. In some embodiments, A2 may be greater than or equal to 80°. In some embodiments, A2 may be greater than or equal to 85°. In some embodiments, A2 may be less than or equal to 105°. In some embodiments, A2 may be less than or equal to 100°. In some embodiments, A2 may be less than or equal to 95°. In some embodiments, A2 may be 180°. In some embodiments, A2 may be greater than or equal to 150°. In some embodiments, A2 may be greater than or equal to 155°. In some embodiments, A2 may be greater than or equal to 160°. In some embodiments, A2 may be greater than or equal to 165°. In some embodiments, A2 may be greater than or equal to 170°. In some embodiments, A2 may be greater than or equal to 175°.

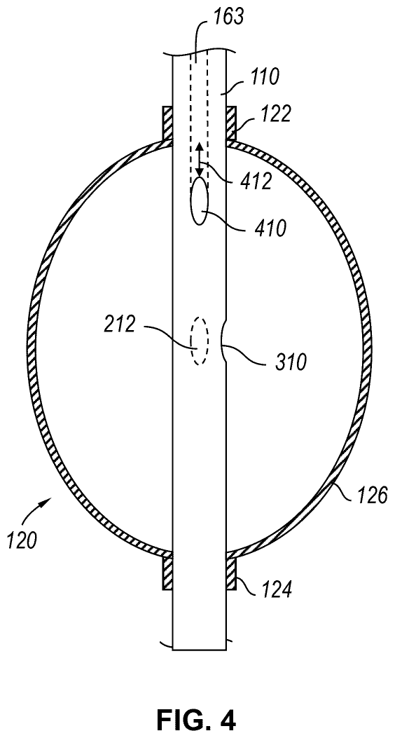

Referring to FIG. 4, a defined exit point 410 of the third lumen 163 may be disposed at a distance 412 from the proximal end of the occlusive element. In some embodiments, the defined exit point 410 may be disposed axially between the defined exit point 310 of the second lumen and the proximal end of the expandable occlusion element. In some embodiments, the defined exit point 410 may be disposed closer to the proximal end of the expandable occlusion element than to the defined exit point 310 of the second lumen.

Similar to what is shown in FIG. 2B, the defined exit point of the third lumen may be rotated circumferentially, relative to any defined fluid exit points of the first lumen, by an angle A3. In some embodiments, A3 may be greater than or equal to 75°. In some embodiments, A3 may be greater than or equal to 150° and less than or equal to 180°. In some embodiments A3 equals 90°. In some embodiments, A3 may be greater than or equal to 75°. In some embodiments, A3 may be greater than or equal to 80°. In some embodiments, A3 may be greater than or equal to 85°. In some embodiments, A3 may be less than or equal to 105°. In some embodiments, A3 may be less than or equal to 100°. In some embodiments, A3 may be less than or equal to 95°. In some embodiments, A3 may be 180°. In some embodiments, A3 may be greater than or equal to 150°. In some embodiments, A3 may be greater than or equal to 155°. In some embodiments, A3 may be greater than or equal to 160°. In some embodiments, A3 may be greater than or equal to 165°. In some embodiments, A3 may be greater than or equal to 170°. In some embodiments, A3 may be greater than or equal to 175°.

As shown in FIG. 4, in some embodiments, the defined exit point 410 of the third lumen may be rotated circumferentially from the defined exit point 310 of the second lumen, by an angle A3′. In some embodiments, A3′ may be greater than or equal to 75°. In some embodiments, A3′ may be greater than or equal to 150° and less than or equal to 180°. In some embodiments A3′ equals 90°. In some embodiments, A3′ may be greater than or equal to 75°. In some embodiments, A3′ may be greater than or equal to 80°. In some embodiments, A3′ may be greater than or equal to 85°. In some embodiments, A3′ may be less than or equal to 105°. In some embodiments, A3′ may be less than or equal to 100°. In some embodiments, A3′ may be less than or equal to 95°. In some embodiments, A3′ may be 180°. In some embodiments, A3′ may be greater than or equal to 150°. In some embodiments, A3′ may be greater than or equal to 155°. In some embodiments, A3′ may be greater than or equal to 160°. In some embodiments, A3′ may be greater than or equal to 165°. In some embodiments, A3′ may be greater than or equal to 170°. In some embodiments, A3′ may be greater than or equal to 175°.

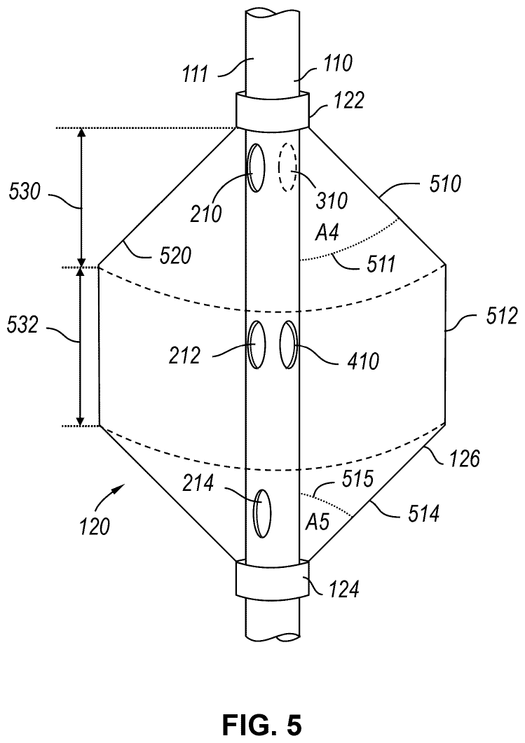

Referring to FIG. 5, while the occlusive element may be any shape, such as oval or circular, in some embodiments, the expandable occlusion element may include a conical distal end 514. In some embodiments, the expandable occlusion element may include a conical proximal end 510. In some embodiments, the expandable occlusion element may include a cylindrical intermediate portion 512 between the distal end 514 and the proximal end 510.

In some embodiments, the expandable occlusion element may define an angle 511 A4 between the external surface 111 of the tubular body and the conical proximal end 510 (such as an inner surface 520 of the expandable occlusion element) when the expandable occlusion element is in its expanded configuration. In some embodiments, A4 may be greater than or equal to 30° and less than or equal to 60°. In some embodiments, A4 may be greater than or equal to 30°. In some embodiments, A4 may be greater than or equal to 35°. In some embodiments, A4 may be greater than or equal to 40°. In some embodiments, A4 may be greater than or equal to 45°. In some embodiments, A4 may be greater than or equal to 50°. In some embodiments, A4 may be greater than or equal to 55°. In some embodiments, A4 may be less than or equal to 60°. In some embodiments, A4 may be less than or equal to 55°. In some embodiments, A4 may be less than or equal to 50°. In some embodiments, A4 may be less than or equal to 45°. In some embodiments, A4 may be less than or equal to 40°. In some embodiments, A4 may be less than or equal to 35°.

In some embodiments, the expandable occlusion element may define an angle 515 A5 between the external surface 111 of the tubular body and the conical distal end 514 (such as an inner surface 520 of the expandable occlusion element) when the expandable occlusion element is in its expanded configuration. In some embodiments, A5 may be greater than or equal to 30° and less than or equal to 60°.

In some embodiments, A5 may be greater than or equal to 30°. In some embodiments, A5 may be greater than or equal to 35°. In some embodiments, A5 may be greater than or equal to 40°. In some embodiments, A5 may be greater than or equal to 45°. In some embodiments, A5 may be greater than or equal to 50°. In some embodiments, A5 may be greater than or equal to 55°. In some embodiments, A5 may be less than or equal to 60°. In some embodiments, A5 may be less than or equal to 55°. In some embodiments, A5 may be less than or equal to 50°. In some embodiments, A5 may be less than or equal to 45°. In some embodiments, A5 may be less than or equal to 40°. In some embodiments, A5 may be less than or equal to 35°. In some embodiments, A4 may equal A5. In some embodiments, A4 may be different from A5.

An axial length 530 of conical proximal end may be longer than an axial length 532 of the cylindrical intermediate portion. An axial length of conical proximal end may be no longer than (e.g., shorter than or equal in length to) an axial length of the cylindrical intermediate portion.

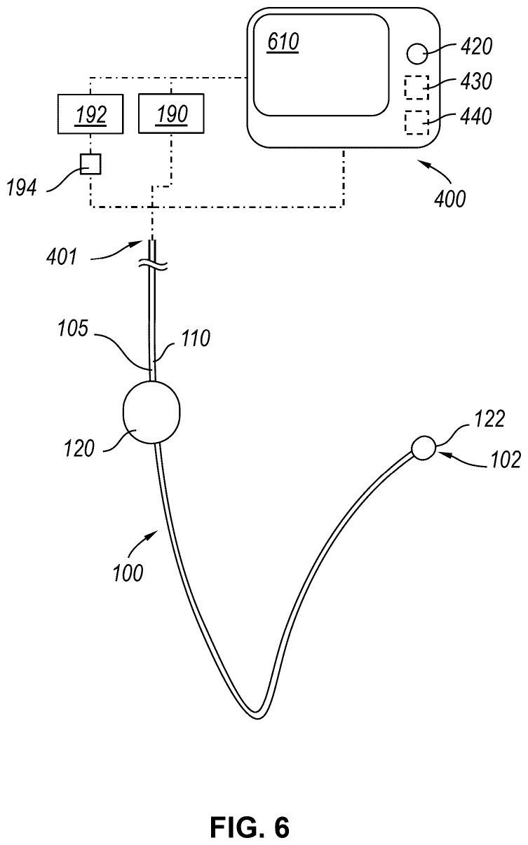

In various aspect a system may be provided. Referring to FIG. 6, the system may include a catheter 105 as disclosed herein.

The system may include a controller 400 (see FIG. 6) operably coupled to the first end 401 of the catheter. The controller may include one or more displays 610 (which may be, e.g., touch-screen displays), one or more knobs or buttons 420, one or more processors 430, and one or more non-transitory computer-readable storage devices 440, which contain instructions that, when executed by the processor(s), cause the processor(s) to operate the device as designed. For example, the processor(s) may be configured to cause the device to selectively occlude, e.g., the superior vena cava via inflating the first balloon for a period of time, and then deflating. Optionally, this could include repeating the inflating/deflating process at a predetermined interval.

The system may include a fluid source 190 operably coupled to the first lumen. The fluid source may include, e.g., a syringe pump configured to dispense a specific amount of a prepared fluid into the occlusion element. The fluid source may be operably coupled to the controller.

The system may include a vacuum pump 192 operably coupled to third lumen. The vacuum pump may be operably coupled to the controller. The system may include a sensor 194, operably coupled to the controller. The sensor may be configured to determine if fluid has passed from the expandable occlusion element through the third lumen. The sensor may be configured to determine how much fluid has passed from the expandable occlusion element through the third lumen.

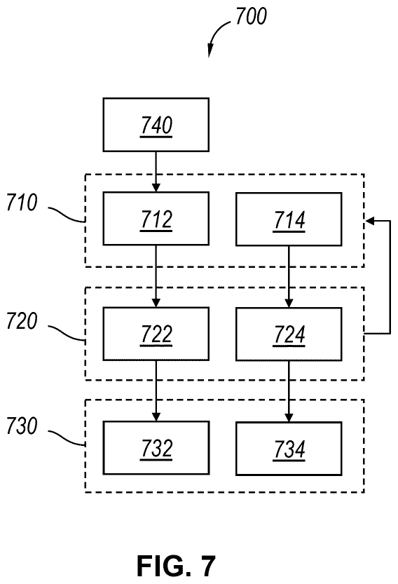

In various aspects, a method for expanding an expandable occlusion element may be provided. Referring to FIG. 7, the method may include controlling 710 the contents of the volume of space within the expandable occlusion element. This may include providing 712 a fluid (such as a saline fluid, which may include a contrast agent) to the expandable occlusion element using one or more inlets. This may include drawing 714 air from the expandable occlusion element using an outlet (e.g., exit point 410 coupled to the third lumen).

In some embodiments, withdrawing air from the expandable occlusion element may occur intermittently (e.g., across the entire filling process, from the time air is first withdrawn to the time air is no longer withdrawn, air is being withdrawn for a first period of time, then stopped for a second period of time, then withdrawn for a third period of time, then stopped, etc.).

In some embodiments, withdrawing fluid (e.g., air) from the expandable occlusion element may occur continuously (e.g., across the entire filling process, from the time air is first withdrawn to the time air is no longer withdrawn, air is being withdrawn continuously). In some embodiments, the withdrawing of fluid (e.g., air) starts when the fluid starts being provided to the expandable occlusion element. In some embodiments, the withdrawing of fluid starts a predetermined period of time after the fluid starts being provided to the expandable occlusion element.

While controlling the contents, the method may include monitoring 720 the process. This may include measuring 722 a pressure within the expandable occlusion element while providing the fluid. This may include determining 724 if fluid has exited the outlet while providing the fluid. That is, initially the vacuum should be removing air from the volume of space, and is ideally leaving all of the fluid used to fill the occlusive element within the volume of space. As such, the method needs to determine if the fluid is exiting the volume of space. In some embodiments, determining if fluid has exited may include determining how much fluid has exited the outlet.

The method may include stopping 730 some or all of the process based on the results of the monitoring step. The method may include stopping 732 fluid provision after determining the pressure has reached a predetermined threshold. This may include stopping 734 withdraw of fluid (e.g., air) based on various criteria. In some embodiments, withdrawing of fluid may be stopped immediately after determining fluid has exited the outlet. In some embodiments, withdrawing of fluid may be stopped, following a predetermined delay, after determining fluid has exited the outlet. In some embodiments, withdrawing of fluid may be stopped after a predetermined amount of fluid has exited the outlet.

The method may include introducing 740 a contrast agent to the fluid. Note, this is shown in FIG. 7 as occurring prior to providing the fluid to the occlusion element, but as will be understood, this can be done at any time until fluid stops being provided to the occlusion element.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

1. A catheter, comprising:
an expandable occlusion element coupled to an external surface of a tubular body at a proximal end of the expandable occlusion element and a distal end of the expandable occlusion element, where an inner surface of the expandable occlusion element defines a volume of space between the inner surface of the expandable occlusion element and an external surface of the tubular body; and
a plurality of lumen, wherein each of the plurality of lumen extends from a proximal end of the tubular body to an external surface of the tubular body at one or more defined exit points between the proximal end of the expandable occlusion element and the distal end of the expandable occlusion element;
wherein the plurality of lumen include:
a first lumen configured to allow a first fluid to be introduced and/or removed from the volume of space defined by the inner surface of the expandable occlusion element;
a second lumen configured to receive a pressure sensor or define a fluid-filled pressure transducer; and
a third lumen configured to allow a second fluid to be removed from the volume of space.

2. The catheter of claim 1, wherein the first fluid comprises a liquid.

3. The catheter of claim 1, wherein the first fluid comprises a saline fluid.

4. The catheter of claim 1, wherein the first lumen is operably coupled to a plurality of defined fluid exit points, each defined fluid exit point being disposed an axial distance from an adjacent defined fluid exit point.

5. The catheter of claim 4, wherein the plurality of defined fluid exit points includes three exit points.

6. The catheter of claim 4, wherein all defined fluid exit points are circumferentially aligned.

7. The catheter of claim 4, wherein at least one defined fluid exit point is rotated circumferentially, relative to an adjacent defined fluid exit point.

8. (canceled)

9. The catheter of claim 1, wherein a defined exit point of the second lumen is centered axially within the volume of space defined by the inner surface of the expandable occlusion element.

10-11. (canceled)

12. The catheter of claim 9, wherein a defined exit point of the third lumen is disposed axially between the defined exit point of the second lumen and the proximal end of the expandable occlusion element.

13-14. (canceled)

15. The catheter of claim 1, wherein the expandable occlusion element is a balloon.

16. The catheter of claim 1, wherein the expandable occlusion element comprises a conical distal end, a conical proximal end, and a cylindrical intermediate portion between the distal end and the proximal end.

17-20. (canceled)

21. The catheter of claim 16, wherein an axial length of conical proximal end is longer than an axial length of the cylindrical intermediate portion.

22. The catheter of claim 16, wherein an axial length of conical proximal end is no longer than an axial length of the cylindrical intermediate portion.

23. The catheter of claim 1, wherein the second fluid is a gas.

24. The catheter of claim 1, wherein the second fluid is air.

25. A system, comprising:
a catheter of claim 1; and
a controller operably coupled to the catheter.

26. The system of claim 25, further comprising a vacuum pump operably coupled to third lumen.

27. (canceled)

28. The system of claim 25, further comprising a sensor, operably coupled to the controller, configured to determine how much first fluid has passed from the expandable occlusion element through the third lumen.

29. A method for expanding an expandable occlusion element, comprising:
providing a first fluid to the expandable occlusion element using one or more inlets and drawing a second fluid from the expandable occlusion element using an outlet;
measuring a pressure within the expandable occlusion element while providing the first fluid and determining if first fluid has exited the outlet while providing the first fluid;
stopping withdraw of the second fluid after determining first fluid has exited the outlet; and
stopping fluid provision after determining the pressure has reached a predetermined threshold.

30-38. (canceled)
